(19) United States
(12) Patent Application Publication
(10) Pub. No.: US 2023/0131340 A1
(43) Pub. Date: Apr. 27, 2023

(54) ULTRASOUND VISCOELASTICITY MEASUREMENT METHOD AND APPARATUS AND STORAGE MEDIUM

(71) Applicant: SHENZHEN MINDRAY BIO-MEDICAL ELECTRONICS CO., LTD., Shenzhen (CN)

(72) Inventors: Shuangshuang LI, SHENZHEN (CN); Jinyang LI, SHENZHEN (CN)

(21) Appl. No.: 17/849,511

(22) Filed: Jun. 24, 2022

Related U.S. Application Data
(63) Continuation of application No. PCT/CN2019/128399, filed on Dec. 25, 2019.

(51) Int. Cl. A61B 8/08; G01S 7/52

(52) U.S. Cl. CPC A61B 8/485; G01S 7/52042; A61B 8/54

(57) ABSTRACT

Disclosed are an ultrasonic viscoelasticity measuring method, an apparatus and a storage medium. The method comprises: outputting a first transmitting/receiving sequence to a transducer of an ultrasonic probe to control the transducer to transmit a first ultrasonic wave to a target object and acquire a first ultrasonic echo signal; generating and displaying an ultrasonic image based on the first ultrasonic echo signal and acquiring a region of interest on the ultrasonic image; outputting different drive signals to a vibrator of the ultrasonic probe to perform viscoelasticity measurement, and exerting various mechanical vibrations on the target object by the transducer driven by the vibrator based on at least two different vibration signals; outputting a second transmitting/receiving sequence to the transducer to control the transducer to transmit a second ultrasonic wave to the region of interest to acquire a second ultrasonic echo signal; and acquiring and displaying elasticity parameter(s) and viscosity parameter(s) of the region of interest based on the second ultrasonic echo signal of the region of interest under the various mechanical vibrations. The proposed scheme can effectively improve the accuracy and stability of measured result.

### TECHNICAL FIELD

The present disclosure relates to transient elasticity measurement, more particularly to ultrasonic viscoelasticity measuring methods, apparatus and storage media.

### BACKGROUND OF THE INVENTION

Hepatic fibrosis is a pathological process that develops from various chronic liver diseases to cirrhosis. Clinically, transient elastography (TE) is used to test the hardness of a liver to reflect the degree of hepatic fibrosis. Compared with the pathological detection of invasive liver biopsy, transient elasticity has the characteristics of non-invasive, simple, rapid, easy to operate, repeatability, safety and good tolerance, thus becoming an important means of clinical evaluation of related hepatic fibrosis.

Transient elastography mainly refers to generating shear waves in tissues through external vibration (such as motor vibration), observing the propagation process of shear waves in tissues through ultrasonic echoes to detect the propagation velocity of shear waves, and further estimating the elastic modulus of tissues, thereby reflecting the degree of hepatic fibrosis. The external vibration of the existing transient elastic imaging methods is constant excitation in which an object to be measured is regarded as conforming to an ideal elastic model. However, most biological tissues often coexist in elasticity and viscosity during deformation, that is, they fail to conform to the ideal elastic model, so such transient elastic imaging method will lead to inaccurate measurement results.

### SUMMARY OF THE INVENTION

The present disclosure provides an ultrasonic viscoelasticity measuring scheme, which can effectively improve the accuracy and stability of measured results by ultrasonic viscoelasticity measurement of targets based on external vibration under different excitation. The ultrasonic viscoelasticity measuring scheme proposed herein is briefly described below, and more details will be described in the specific embodiments in combination with the accompanying drawings.

An ultrasonic viscoelasticity measuring method provided in accordance with an aspect of the present disclosure may include: outputting a first transmitting/receiving sequence to a transducer of an ultrasonic probe to control the transducer to transmit a first ultrasonic wave to a target object, receive an echo of the first ultrasonic wave, and acquire a first ultrasonic echo signal based on the echo of the first ultrasonic wave; generating an ultrasonic image based on the first ultrasonic echo signal and displaying the ultrasonic image, and acquiring a region of interest on the ultrasonic image; outputting different drive signals to a vibrator of the ultrasonic probe to drive the transducer by the vibrator to exert various mechanical vibrations on the target object based on at least two different vibration signals; outputting a second transmitting/receiving sequence to the transducer to control the transducer to transmit a second ultrasonic wave to the region of interest, receive an echo of the second ultrasonic wave, and acquire a second ultrasonic echo signal based on the echo of the second ultrasonic wave; and acquiring and displaying elasticity parameter(s) and viscosity parameter(s) of the region of interest based on the second ultrasonic echo signal of the region of interest under the various mechanical vibrations.

An ultrasonic viscosity measuring method provided in accordance with another aspect of the present disclosure may include: acquiring and displaying a tissue image of a target object; detecting a region of interest selected by a user on the tissue image; exerting various mechanical vibrations on the target object based on at least two different vibration signals to generate a shear wave within the region of interest; transmitting an ultrasonic wave to the region of interest after the mechanical vibrations are generated, receiving an echo of the ultrasonic wave, and acquiring an ultrasonic echo signal based on the echo of the ultrasonic wave; and acquiring and displaying at least one of elasticity parameter(s) and viscosity parameter(s) of the region of interest based on the ultrasonic echo signal within the region of interest under the various mechanical vibrations.

An ultrasonic viscoelasticity measuring method provided in accordance with yet another aspect of the present disclosure may include: exerting various mechanical vibrations on a target object based on at least two different vibration signals; transmitting an ultrasonic wave to the target object, receiving an echo of the ultrasonic wave, and acquiring an ultrasonic echo signal based on the echo of the ultrasonic wave; and acquiring elasticity parameter(s) and viscosity parameter(s) of the target object based on the ultrasonic echo signal of the target object under the various mechanical vibrations.

An ultrasonic viscoelasticity measuring apparatus provided in accordance with still yet another aspect of the present disclosure may include: an ultrasonic probe comprising a vibrator and a transducer, the vibrator being configured for driving the transducer to vibrate to generate a shear wave propagating in a depth direction inside a target object; the transducer comprising a plurality of array elements, at least part of the array elements being configured for transmitting a first ultrasonic wave to the target object, receiving an echo of the first ultrasonic wave and acquiring a first ultrasonic echo signal based on the echo of the first ultrasonic wave before the transducer is vibrated, and at least transmitting a second ultrasonic wave to a region of interest of the target object, receiving an echo of the second ultrasonic wave and acquiring a second ultrasonic echo signal based on the echo of the second ultrasonic wave after the transducer is vibrated; a transmitting/receiving sequence controller configured for outputting a first transmitting/receiving sequence to the transducer before the transducer is vibrated to control the transducer to transmit the first ultrasonic wave, receive the echo of the first ultrasonic wave and acquire the first ultrasonic echo signal based on the echo of the first ultrasonic wave, outputting different drive signals to the vibrator after the region of interest is determined to control the vibrator to drive the transducer to exert various mechanical vibrations on the target object based on at least two different vibration signals, and at least outputting a second transmitting/receiving sequence to the transducer after the transducer is vibrated to control the transducer to transmit the second ultrasonic wave, receive the echo of the second ultrasonic wave and acquire the second ultrasonic echo signal based on the echo of the second ultrasonic wave; a processor configured for generating an ultrasonic image based on the first ultrasonic echo signal, acquiring a region of interest on the ultrasonic image, and acquiring elasticity parameter(s) and viscosity parameter(s) of said region of interest based on the second ultrasonic echo signal of the region of interest under various mechanical vibrations; and a display unit configured for displaying the elasticity parameter(s) and the viscosity parameter(s) of said region of interest.

An ultrasonic viscoelasticity measuring apparatus provided in accordance with yet still yet another aspect of the present disclosure may include: an ultrasonic probe comprising a vibrator and a transducer, the vibrator being configured for driving the transducer to vibrate to generate a shear wave propagating in a depth direction inside a target object; the transducer comprising one or more array elements, at least part of the array elements being configured for at least after the transducer is vibrated, transmitting an ultrasonic wave to a region of interest of the target object, receiving an echo of the ultrasonic wave and acquiring an ultrasonic echo signal based on the echo of the ultrasonic wave; a transmitting/receiving sequence controller configured for after the region of interest is determined outputting different drive signals to the vibrator to control the vibrator to drive the transducer to exert various mechanical vibrations on the target object based on at least two different vibration signals, and at least after the transducer is vibrated, outputting a transmitting/receiving sequence to the transducer to control the transducer to transmit the ultrasonic wave, receive the echo of the ultrasonic wave and acquire the ultrasonic echo signal based on the echo of the ultrasonic wave; a processor configured for acquiring a tissue image of the target object, acquiring a region of interest on the tissue image, and acquiring elasticity parameter(s) and viscosity parameter(s) of said region of interest based on the ultrasonic echo signal of the region of interest under the various mechanical vibrations; and a human-machine interactive unit configured for detecting the region of interest selected by a user on the tissue image, and displaying the elasticity parameter(s) and the viscosity parameter(s) of said region of interest.

An ultrasonic viscoelasticity measuring apparatus provided in accordance with another aspect of the present disclosure may include: a vibrator, an ultrasonic probe, a scanning controller and a processor, wherein: the vibrator is configured for exerting various mechanical vibrations on a target object based on at least two different vibration signals; the scanning controlling is configured for exciting the ultrasonic probe to transmit an ultrasonic wave to the target object, receive an echo of the ultrasonic wave and acquire an ultrasonic echo signal based on the echo of the ultrasonic wave; and the processor is configured for acquiring elasticity parameter(s) and viscosity parameter(s) of the target object based on the ultrasonic echo signal of the target object under the various mechanical vibrations.

An ultrasonic viscosity measuring apparatus provided in accordance with yet still yet another aspect of the present disclosure may include: a processor and a memory storing a computer program run by the processor, wherein the computer program may execute the ultrasonic viscoelasticity measuring method mentioned above when being run by the processor.

A storage medium provided in accordance with still another aspect of the present disclosure may store a computer program that may execute the ultrasonic viscoelasticity measuring method mentioned above when being run.

With the ultrasonic viscoelasticity measuring method, apparatus and storage medium according to embodiments of the present disclosure, ultrasonic viscoelasticity measurement may be performed on a target object on the basis of an external vibration under different excitation, which can obtain elasticity parameter(s) and viscous parameter(s) of a region of interest of the target object, solving the problem of inaccurate and unstable measurement when using an ideal elasticity model, and improving the accuracy and stability of the measurement.

### BRIEF DESCRIPTION OF THE DRAWINGS

FIG. 1 is a schematic diagram of a transient elasticity imaging method;

FIG. 2 is a schematic diagram showing “dispersion” of measured elasticity values in pure elasticity model under different excitation;

FIG. 3 is a schematic diagram of measured elasticity values and measured viscosity values in a viscoelasticity model under different excitation;

FIG. 4 is a schematic diagram of a simplified viscoelasticity model;

FIG. 5 is a schematic flowchart of an ultrasonic viscoelasticity measuring method according to an embodiment of the present disclosure;

FIG. 6 is a schematic flowchart of performing a plurality of measurements on a target object in an ultrasonic viscoelasticity measuring method according to an embodiment of the present disclosure;

FIG. 7 is a schematic flowchart of an ultrasonic viscoelasticity measuring method according to another embodiment of the present disclosure;

FIG. 8 is a schematic flowchart of an ultrasonic viscoelasticity measuring method according to yet another embodiment of the present disclosure;

FIG. 9 is a schematic block diagram of an ultrasonic viscoelasticity measuring apparatus according to an embodiment of the present disclosure;

FIG. 10 is a schematic block diagram of an ultrasonic viscoelasticity measuring apparatus according to another embodiment of the present disclosure;

FIG. 11 is a schematic block diagram of an ultrasonic viscoelasticity measuring apparatus according to yet another embodiment of the present disclosure;

FIG. 12 is a schematic diagram of a system frame in which an ultrasonic viscoelasticity measuring apparatus according to an embodiment of the present disclosure performs ultrasonic viscoelasticity measurement; and

FIG. 13 is a schematic block diagram of an ultrasonic viscoelasticity measuring apparatus according to still another embodiment of the present disclosure.

### DETAILED DESCRIPTION

In order to make the objectives, technical solutions, and advantages of the present disclosure clearer, example embodiments according to the present disclosure will be described in detail below with reference to the accompanying drawings. Apparently, the described embodiments are merely some rather than all of the embodiments of the present disclosure. It should be understood that the example embodiments described herein do not constitute any limitation to the present disclosure. All other embodiments derived by those skilled in the art without creative efforts on the basis of the embodiments of the present disclosure described in the present disclosure shall fall within the scope of protection of the present disclosure.

In the following description, a large number of specific details are given to provide a more thorough understanding of the present disclosure. However, it would be understood by those skilled in the art that the present disclosure can be implemented without one or more of these details. In other examples, to avoid confusion with the present disclosure, some technical features known in the art are not described.

It should be understood that the present disclosure can be implemented in different forms and should not be construed as being limited to the embodiments presented herein. On the contrary, these embodiments are provided to make the disclosure thorough and complete, and to fully convey the scope of the present disclosure to those skilled in the art.

The terms used herein are intended only to describe specific embodiments and do not constitute a limitation to the present disclosure. When used herein, the singular forms of “a”, “an”, and “said/the” are also intended to include plural forms, unless the context clearly indicates otherwise. It should also be appreciated that the terms “comprise” and/or “include”, when used in the specification, determine the existence of described features, integers, steps, operations, elements, and/or units, but do not exclude the existence or addition of one or more other features, integers, steps, operations, elements, units, and/or groups thereof. As used herein, the term “and/or” includes any and all combinations of relevant listed items.

For a thorough understanding of the present disclosure, detailed steps and detailed structures will be provided in the following description to explain the technical solutions proposed by the present disclosure. The preferred embodiments of the present disclosure are described in detail as follows. However, in addition to these detailed descriptions, the present disclosure may further have other implementations.

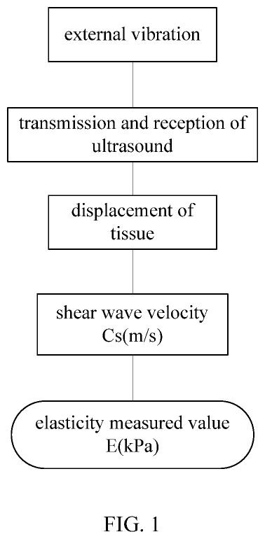

The main principle of transient elastography, as shown in FIG. 1, mainly refers to generating shear waves in tissues through external vibration (such as motor vibration), observing the propagation process of shear waves in tissues through ultrasonic echoes to detect the propagation velocity of shear waves, and further estimating the elastic modulus of tissues. In FIG. 1, the external vibration may be equivalent to a “signal source” of the shear waves, the shear waves generated by the excitation thereof and propagating in the tissues plays a decisive role in final elasticity measurement. In existing transient elastography, the external vibration is constant, which not only has certain requirements in terms of test conditions, but also requires certain assumptions about a test object, that is, the test object conforms to an ideal elasticity model.

Mechanical models includes both elasticity and viscosity. In an ideal elasticity model, stress obeys Hooke's law and only depends on strain which recovers after the removal of an external force; and corresponding substance involved is called Hooke solid. In an ideal viscosity model, stress obeys Newton's fluid law and only depends on strain rate, strain changes with time, deformation generated is irreversible, and corresponding substance involved is called Newton's liquid. For most materials, including biological soft tissues, elasticity and viscosity tend to coexist during deformation, wherein stress depends on both deformation and deformation velocity at the same time, resulting in having both solid and liquid Characteristics which are between ideal elasticity and ideal viscosity. Such property is called viscoelasticity.

For the clinical application of transient elasticity, the existing transient elastography only focuses on elasticity measurement. However, the viscosity of biological tissues can also provide a lot of tissue information.

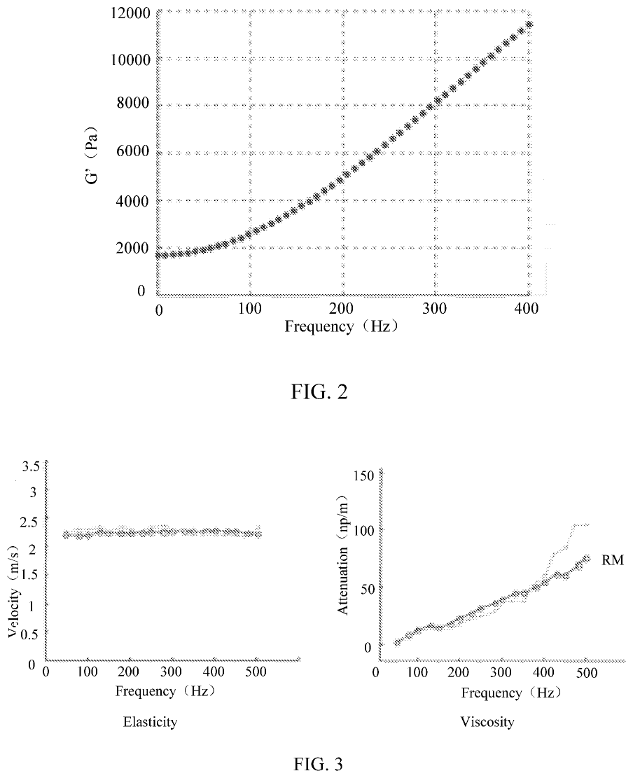

For transient elasticity measurement, a test object (such as liver) is regarded as the ideal elasticity model in the existing transient elastography, which leads to obvious differences and certain rules in measured elasticity values under different excitation of external vibrations, which is called “dispersion”, as shown in FIG. 2. The reason for this phenomenon is that the model is too ideal to match an actual situation, which increases the instability of the measured result to a certain extent. The applicant found that if the viscoelasticity model is to be used, it can be seen that both elasticity and viscosity show relatively stable performance under different excitation, as shown in FIG. 3.

Under the ideal elasticity model, the elasticity measurement is usually only related to phase information, and an elastic coefficient μ and a shear wave velocity v may be simply expressed as the following formula (1):

μ=3ρv² formula (1)

where ρ represents density.

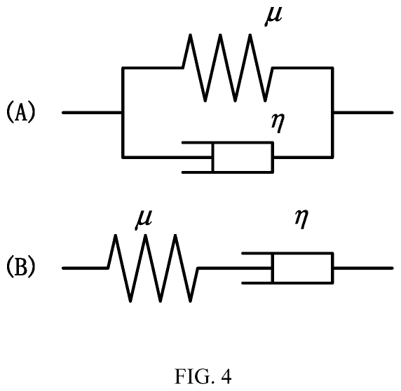

In addition to paying attention to the phase information of the shear waves, the amplitude information of the shear waves may also be needed in viscoelasticity measurement, which can be divided into two simplified models, as shown in FIG. 4(A) and FIG. 4(B). The relationship among elastic coefficient μ and viscosity coefficient η of the two models together with the velocity v and attenuation α of the shear waves at different frequencies ω under ideal conditions can be expressed as the following formula (2) and formula (3):

$$\begin{matrix}{v}_{A} = \sqrt{\frac{2 ( {μ}^{2} + {ω}^{2} {η}^{2} )}{ρ ( μ + \sqrt{{μ}^{2} + {ω}^{2} {η}^{2}} )}} & {α}_{A} = \sqrt{\frac{2 μ}{ρ ( 1 + \sqrt{1 + \frac{{μ}^{2}}{{ω}^{2} {η}^{2}}} )}}\end{matrix} \qquad formula ( 2 )$$

$$\begin{matrix}{v}_{B} = \sqrt{\frac{{ρω}^{2} \sqrt{( {μ}^{2} + {ω}^{2} {η}^{2} ) - μ}}{2 ( {μ}^{2} + {ω}^{2} {η}^{2} )}} & {α}_{B} = \sqrt{\frac{{ρω}^{2} ( \sqrt{1 + \frac{{μ}^{2}}{{ω}^{2} {η}^{2}} - 1} )}{2 μ}}\end{matrix} \qquad formula ( 3 )$$

Regardless of the model, corresponding viscosity coefficient and elasticity coefficient can be estimated from the shear wave information of multiple frequencies.

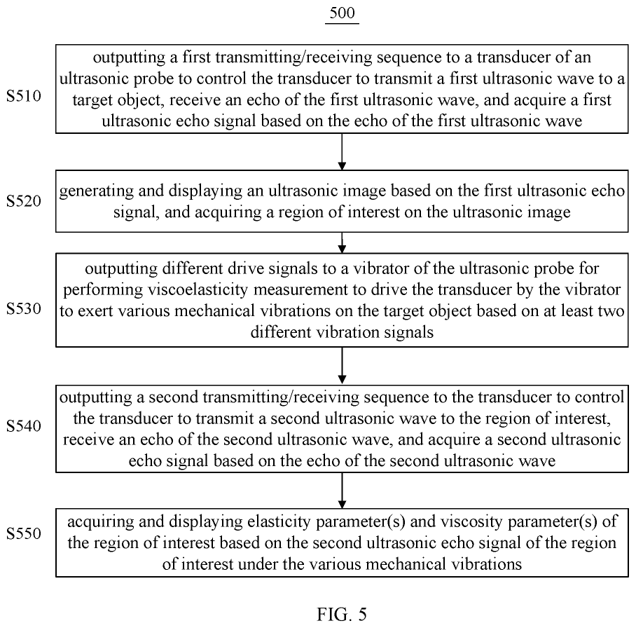

In view of the above description, with an ultrasonic viscoelasticity measuring method provided in the present disclosure, the ultrasonic viscoelasticity measurement may be performed on a target based on external vibrations under various excitation, effectively improving the accuracy and stability of measured result. The ultrasonic viscoelasticity measuring method according to the present disclosure is described in detail below with reference to FIGS. 5-13.

FIG. 5 shows an ultrasonic viscoelasticity measuring method 500 according to an embodiment of the present disclosure. As shown in FIG. 5, the ultrasonic viscoelasticity measuring method 500 may include the following steps:

Step S510: outputting a first transmitting/receiving sequence to a transducer of an ultrasonic probe to control the transducer to transmit a first ultrasonic wave to a target object, receive an echo of the first ultrasonic wave, and acquire a first ultrasonic echo signal based on the echo of the first ultrasonic wave.

In an embodiment of the present disclosure, outputting the first transmitting/receiving sequence to the transducer of the ultrasonic probe is for obtaining an ultrasonic image. Based on the first transmitting/receiving sequence, the transducer of the ultrasonic probe may transmit the first ultrasonic wave to the target object (such as a biological tissue), and convert a received echo of the first ultrasonic wave into electrical signal, thereby acquiring a first ultrasonic echo signal. It should be noted that the “first transmitting/receiving sequence”, the “first ultrasonic wave” and the “first ultrasonic echo signal” herein are so named only to distinguish them from a “second transmitting/receiving sequence”, a “second ultrasonic wave” and a “second ultrasonic echo signal” mentioned below, without any restrictive meaning.

Step S520: generating an ultrasonic image based on the first ultrasonic echo signal and displaying the ultrasonic image, and acquiring a region of interest on the ultrasonic image;

In an embodiment of the present disclosure, the first ultrasonic echo signal acquired in S510 may be processed to generate an ultrasonic image data, such as B image data, C image data, or a superposition of the two. Based on the generated ultrasound image data, the ultrasound image can be obtained. In one example, the region of interest of the target object (such as a region corresponding to a liver to be measured for viscoelasticity) may be automatically detected on the ultrasonic image based on a relevant algorithm to acquire the region of interest. In another example, the ultrasonic image may be displayed to let a user to manually select the region of interest of the target object on the ultrasonic image; in this respect, a user input may be checked to acquire the region of interest selected by the user. In other examples, the region of interest may also be acquired via semi-automatic detection. In the semi-automatic detection, a rough region may be selected first by the user, and then a more accurate region within the rough region selected by the user may be automatically detected based on a certain algorithm to obtain the region of interest. Alternately, in the semi-automatic detection, the region of interest on the ultrasonic image may first be automatically detected based on a certain algorithm, and then be modified or corrected by the user to obtain a more accurate region of interest.

Step S530: outputting different drive signals to a vibrator of the ultrasonic probe to drive the transducer by the vibrator to exert various mechanical vibrations on the target object based on at least two different vibration signals.

In the embodiment of the present disclosure, the ultrasonic probe itself including a vibrator is described as an example. However, it should be understood that the vibrator may also be independent of the ultrasonic probe. When the ultrasonic probe itself is provided with the vibrator, a drive signal for driving the vibrator to vibrate may be outputted to the vibrator of the ultrasonic probe for viscoelasticity measurement. In the embodiment of the present disclosure, instead of using a fixed drive signal (i.e. a fixed excitation) to drive the vibrator for measurement, different drive signals may be used to drive the vibrator for measurement. Different drive signals outputted by the vibrator make the vibrator to exert different mechanical vibrations on the target object based on at least two different vibration signals. Exemplarily, the difference among the vibration signals may be shown as follows: different vibration signals being different from each other in vibration waveform; different vibration signals being different from each other in different frequencies; or any other possible difference. Using different drive signals to drive the vibrator to perform viscoelasticity measurement may enable the vibrator to exert different mechanical vibrations under different vibration signals, thus obtaining a shear wave data of the region of interest of the target object under different mechanical vibrations. Further, based on the shear wave data of the region of interest of the target object under different mechanical vibrations, a more stable and accurate measured result about elasticity and viscosity can be obtained.

Step S540: outputting a second transmitting/receiving sequence to the transducer to control the transducer to transmit a second ultrasonic wave to the region of interest, receive an echo of the second ultrasonic wave, and acquire a second ultrasonic echo signal based on the echo of the second ultrasonic wave.

In an embodiment of the present disclosure, outputting the second transmitting/receiving sequence to the transducer of the ultrasonic probe is for detecting viscoelasticity of the region of interest. Based on the second transmitting/receiving sequence, the transducer of the ultrasonic probe may transmit the second ultrasonic wave to the target object, and convert a received echo of the second ultrasonic wave into electrical signal, thereby acquiring a second ultrasonic echo signal. As mentioned above, the “second transmitting/receiving sequence”, the “second ultrasonic wave” and the “second ultrasonic echo signal” herein are so named only to distinguish them from the “first transmitting/receiving sequence”, the “first ultrasonic wave” and the “first ultrasonic echo signal” mentioned above, without any restrictive meaning.

In an embodiment of the present disclosure, the transducer may output the second transmitting/receiving sequence after the mechanical vibrations are generated by the vibrator to perform ultrasonic scanning on the region of the interest. In other examples, the transducer may outputting the second transmitting/receiving sequence before the vibrator generates the mechanical vibrations (for example outputting after the region of interest is determined) to perform ultrasonic scanning on the region of interest. In other examples, the transducer may also output the second transmitting/receiving sequence at the same time when the vibrator generates the mechanical vibrations.

Step S550: acquiring and displaying elasticity parameter(s) and viscosity parameter(s) of the region of interest based on the second ultrasonic echo signal of the region of interest under various mechanical vibrations.

In an embodiment of the present disclosure, the second ultrasonic echo signal of the region of interest under various mechanical vibrations may be processed separately to obtain measured elasticity values and measured viscosity values of the region of interest under different mechanical vibrations, and a final measured result of elasticity (i.e. the elasticity parameter(s)) and that of viscosity (i.e. the viscosity parameter(s)) of the region of interest may be acquired based on the measured elasticity values and the measured viscosity values. For example, an average value, weighted average value, any value, minimum value, maximum value, or average value of any number of values of all measured elasticity values may be taken as a final measured result of elasticity as required. Similarly, for example, an average value, weighted average value, any value, minimum value, maximum value, of average value of any number of values of all measured viscosity values may be taken as a final measured result of viscosity as required. Alternatively, the measured elasticity values and the measured viscosity values may be directly taken as a final measured result of viscoelasticity.

For example, assuming that the vibrator output M (M≥2) different mechanical vibrations, one elasticity measured data and one viscosity measured data may be calculated based on the second ultrasonic echo signal of the region of interest under each mechanical vibration, in this respect, a plurality of elasticity measured data and a plurality of viscosity measured data may be obtained by repeating the calculation for M times based on the second ultrasonic echo signal. In an embodiment of the present disclosure, the plurality of elasticity measured data may be made statistics, such as calculating the average value, weighted average value, any value, minimum value, maximum value, or average value of any number of values of the plurality of elasticity measured data, and a statistical result thereof may be taken as the measured elasticity value. In an embodiment of the present disclosure, the measured viscosity value may be calculated based on at least two viscosity measured data from the plurality of viscosity measured data. For example, with reference to the viscosity shown in FIG. 3, a slope may be determined based on at least two viscosity measured data, and the value of the slope may be taken as the measured viscosity value. In some examples, a difference or ratio between at least two viscosity measuring data may be calculated and the difference or ratio may be taken as the measured viscosity value.

The viscoelasticity measurement in different examples based on the above method is described in detail below.

In an example, the target object may be performed with one measurement. Such measurement may be implemented by exerting mechanical vibrations on the target object based on different vibration signals, wherein each vibration signal corresponds to one ultrasonic echo signal; and the elasticity parameter(s) and the viscosity parameter(s) of the region of interest may be acquired by calculating a group of measured elasticity value and measured viscosity value based on a plurality of ultrasonic echo signals corresponding to the plurality of different vibration signals, thereby acquiring the elasticity parameter and the viscosity parameter respectively based on the group of measured elasticity value and measured viscosity value. In an embodiment of the present disclosure, “one measurement” may, from clinical operation, be defined as being measured by a user by pressing a button once or inputting an instruction once or other operation once. In this respect, in this example, the user may obtain a group of elasticity parameter and viscosity parameter only with simple operation.

In another example, the target object may be performed with one measurement that includes multiple groups of sub-measurements, wherein in each group of sub-measurement, the target object may be exerted with mechanical vibrations based on a plurality of different vibration signals, each vibration signal corresponds to one ultrasonic echo signals; and the elasticity parameter(s) and the viscosity parameter(s) of the region of interest may be acquired by calculating to acquire multiple groups of elasticity parameters and viscosity parameters based on the plurality of ultrasonic echo signals corresponding to the plurality of different vibration signals in each group of sub-measurement. In this example, the user may still only need to press the button once or input the instruction once in other ways; however, unlike the aforesaid example, this measurement includes multiple groups of sub-measurements, and multiple groups of measured elasticity values and multiple groups of measured viscosity values obtained directly based on the multiple groups of sub-measurements may be taken as the measured result of viscoelasticity, thus the multiple groups of elasticity parameters and viscosity parameters can be measured.

In yet another example, the target object may be performed with one measurement that includes multiple groups of sub-measurements, wherein in each group of sub-measurement, the target object may be exerted with mechanical vibrations based on a plurality of different vibration signals, each vibration signal corresponds to one ultrasonic echo signals; and the elasticity parameter(s) and the viscosity parameter(s) of the region of interest may be acquired by calculating the elasticity parameter(s) and the viscosity parameter(s) based on multiple groups of measured elasticity values and measured viscosity values, where each group of measured elasticity value and measured viscosity value is calculated based on a plurality of ultrasonic echo signals corresponding to the plurality of different vibration signals in each group of sub-measurement. In this example, the user may still only need to press the button once or input the instruction once in other ways; however, unlike the previous example, this measurement includes multiple groups of sub-measurements, and the result of the viscoelasticity in this example is further calculated based on the multiple groups of measured elasticity values and measured viscosity values, thereby acquiring a more accurate measured result of the elasticity parameter(s) and the viscosity parameter(s).

Exemplarily, the multiple groups of sub-measurements may be the ones that perform measuring continuously in one measurement. Said measuring continuously may refer to that after a previous group of sub-measurement is completed, a next group of sub-measurement may be started automatically after a predetermined time interval without a startup command inputted again by the user between the two groups of sub-measurements. Exemplarily, the target object may be exerted with the same number of mechanical vibrations in each group of sub-measurement. Exemplarily, a group of different vibration signals may be generated based on the same drive signal in each group of sub-measurement. For each group of sub-measurement, with exerting mechanical vibrations of the same number to the target object, and/or with generation of a group of different vibration signals based on the same drive signal, each group of sub-measurement can be performed under the same external conditions, thereby obtaining a more accurate measured result.

In other examples, during performing a plurality of sub-measurement on the target object, the number and/or waveforms of the vibration signals used in each sub-measurement may be different. Exemplarily, during each measurement performed on the target object, at least one of the following parameters of each drive signal for the plurality of different vibration signals is different: frequency, amplitude, phase and the number of periods, and at least one of the following parameters of the different vibration signals is different: frequency, amplitude, phase and the number of periods. In general, the drive signals and actual vibration waveform may be unequal, and there may be a differential relationship therebetween in the ideal model.

In still another example, the target object may be performed with a plurality of measurements, wherein in each of the plurality of measurements, mechanical vibrations may be exerted on the target object based on a plurality of different vibration signals, each vibration signal corresponds to one ultrasonic echo signal; and the elasticity parameter(s) and the viscosity parameter(s) of the region of interest may be acquired by calculating out multiple groups of elasticity parameters and viscosity parameters based on a plurality of ultrasonic echo signals corresponding to the multiple different vibration signals in each measurement. That is, a group of measured result of the elasticity parameter(s) and the viscosity parameter(s) may be outputted in each measurement. In this example, “a plurality of measurements” may, from clinical operation, be defined as being measured by a user by pressing a button for multiple times or inputting an instruction for multiple times or other operations for multiple times. Based on this, in this example, the user may obtain multiple groups of measured elasticity values and measured viscosity values after multiple operations and finally obtain multiple groups of elasticity parameters and viscosity parameters based on the multiple groups of measured elasticity values and measured viscosity values.

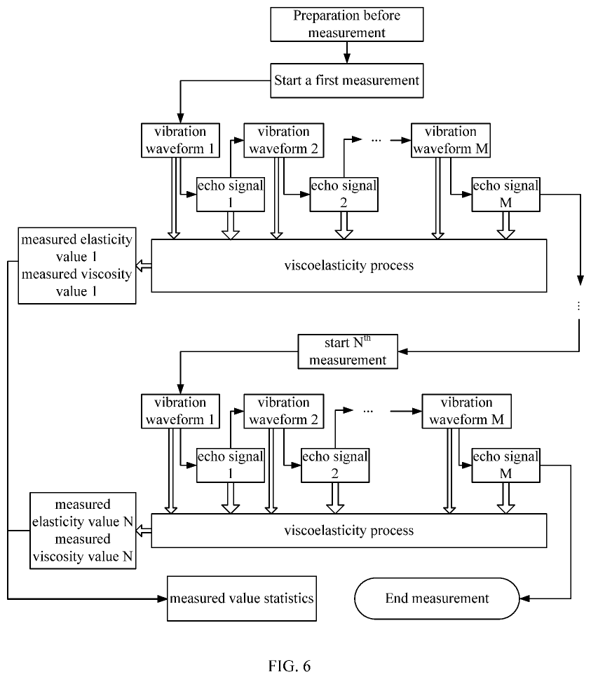

In yet still another example, the target object may be performed with a plurality of measurements, wherein in each of the plurality of measurements, mechanical vibrations may be exerted on the target object based on a plurality of different vibration signals, each vibration signal corresponds to one ultrasonic echo signal; and the elasticity parameter(s) and the viscosity parameter(s) of the region of interest may be acquired by calculating the elasticity parameter(s) and the viscosity parameter(s) based on multiple groups of measured elasticity values and measured viscosity values, where each group of the measured elasticity value and the measured viscosity value is calculated based on a plurality of ultrasonic echo signals acquired in each measurement. In this example, “a plurality of measurements” may, from clinical operation, be defined as being measured by a user by pressing a button for multiple times or inputting an instruction for multiple times or other operations. Based on this, in this example, the user may obtain multiple groups of measured elasticity values and measured viscosity values after multiple operations and finally obtain multiple groups of elasticity parameters and viscosity parameters based on the multiple groups of measured elasticity values and measured viscosity values. The process of the aforesaid plurality of measurements may be understood with reference to FIG. 6. In FIG. 6, it is shown by example that the measurement may be performed for N times (where N is a natural number), M vibration waveforms (where M is a natural number) may be adopted in each measurement, and N groups of measured elasticity values and measured viscosity values may be obtained. Then a final measured result may be acquired by making statistics on the measured values.

Exemplarily, during performing the plurality of measurements on the target object, the number and/or waveforms of the vibration signals used in each measurement may be different. Exemplarily, during each measurement performed on the target object, at least one of the following parameters of each drive signal for the plurality of different vibration signals may be different: frequency, amplitude, phase and the number of periods; and at least one of the following parameters of the different vibration signals may be different: frequency, amplitude, phase and the number of periods. In general, the drive signal and actual vibration waveform may be unequal, and there may be a differential relationship therebetween in the ideal model.

In still yet another example, the target object may be performed with a plurality of measurements, wherein in each of the plurality of measurements, mechanical vibrations may be exerted on the target object based on a single vibration signal, the vibration signals in each measurement may be different in the plurality of measurements and may correspond to one ultrasonic echo signal; and the elasticity parameter(s) and the viscosity parameter(s) of the region of interest may be acquired by calculating out a group of elasticity parameter and viscosity parameter based on a plurality of ultrasonic echo signals corresponding to multiple different vibration signals of the plurality of measurements. In an embodiment of the present disclosure, “a plurality of measurements” may, from clinical operation, be defined as being measured by a user by pressing a button for multiple times or inputting an instruction for multiple times or other operations for multiple times. Based on this, in this example, the user may obtain a group of measured elasticity value and measured viscosity value after performing multiple operations and finally obtain multiple groups of elasticity parameters and viscosity parameters based on the group of measured elasticity value and measured viscosity value, for example the group of measured elasticity value and measured viscosity value may be taken as the elasticity parameter and the viscosity parameter.

In an embodiment of the present disclosure, the target object may be performed with each measurement based on a received instruction inputted by a user that at least includes viscoelasticity measurement, or based on other preset conditions. In addition, for example, in each measurement, after mechanical vibrations may be exerted on the target object based on one vibration signal to obtain a corresponding ultrasonic echo signal, the target object may be exerted with mechanical vibrations based on another vibration signal after a predetermined cooling time, thereby acquiring a more accurate measured result.

In another embodiment of the present disclosure, the acquired measured results of elasticity and viscosity may be displayed. Exemplarily, each group of measured elasticity value and measured viscosity value may be displayed, or only the measured results of elasticity and viscosity that are calculated respectively based on the measured elasticity values and the measured viscosity values may be displayed. Further, the ultrasonic image may be displayed while displaying the elasticity parameter(s) and the viscosity parameter(s) of the region of interest. The ultrasonic image may be generated based on the first ultrasonic echo signal or the second ultrasonic echo signal. The ultrasonic image may be acquired in real time during viscoelasticity measurement, or be acquired at certain intervals during viscoelasticity measurement, or be a non-real-time image that is acquired and not updated before each viscoelasticity measurement. For example, the elasticity parameter(s)/measured value(s) and the viscosity parameter(s)/measured value(s) of the region of interest may be displayed at an appropriate location in the ultrasonic image (e.g. at lower right corner or at middle of region of interest). For example, the elasticity parameter(s)/measured value(s) and the viscosity parameter(s)/measured value(s) of the region of interest may be displayed in a non-image region near the image on the display, such as they may be side by side with the ultrasonic image.

The above examples shows the ultrasonic viscoelasticity measuring method 500 according to an embodiment of the present disclosure. Based on the above description, with the ultrasonic viscoelasticity measuring method 500 according to embodiments of the present disclosure, the ultrasonic viscoelasticity measurement may be performed on a target object based on external vibrations under different excitation, which can obtain elasticity parameter(s) and viscous parameter(s) of a region of interest of the target object, solving the problem of inaccurate and unstable measurement when using the ideal elasticity model, and improving the accuracy and stability of the measurement.

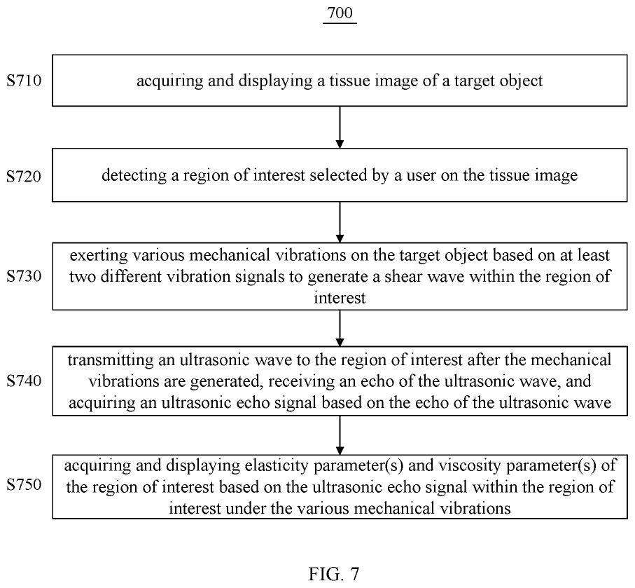

The ultrasonic viscoelasticity measuring method according to another embodiment of the present disclosure is described below with reference to FIG. 7. FIG. 7 shows a schematic flowchart of the ultrasonic viscoelasticity measuring method 700 according to another embodiment of the present disclosure. As shown in FIG. 7, the ultrasonic viscoelasticity measuring method 700 may include the following steps:

Step S710: acquiring and displaying a tissue image of a target object;

Step S720: detecting a region of interest selected by a user on the tissue image;

Step S730: exerting various mechanical vibrations on the target object based on at least two different vibration signals to generate a shear wave within the region of interest;

Step S740: after the mechanical vibrations are generated, transmitting an ultrasonic wave to the region of interest, receiving an echo of the ultrasonic wave, and acquiring an ultrasonic echo signal based on the echo of the ultrasonic wave; and

Step S750: acquiring and displaying at least one of elasticity parameter(s) and viscosity parameter(s) of the region of interest based on the ultrasonic echo signal within the region of interest under the various mechanical vibrations.

There are only slight difference between the ultrasonic viscosity and/or elasticity measuring method 700 described with reference to FIG. 7 according to another embodiment of the present disclosure and the ultrasonic viscoelasticity measuring method 500 described with reference to FIG. 5 according to the embodiment of the present disclosure. For simplicity, the same details will not be repeated here. In the embodiment described in FIG. 7, the tissue image of the target object may be any image that can reflect the tissue structure, such as an ultrasonic image, an MRI image, or a CT image; and the tissue image of the target object may be acquired in real time or from the storage medium of an ultrasonic imaging system or from the storage medium of other external devices. In addition, in the embodiment described with reference to FIG. 7, the region of interest on the tissue image may be acquired based on a user input for generating the shear wave within the region of interest. In the embodiment described in FIG. 7, the ultrasonic probe used may be a single array element and the ultrasonic echo signal obtained in S740 may correspond to M data; alternatively, the ultrasonic probe used may be a plurality of array elements and the ultrasonic echo signal obtained in S740 may correspond to M data or B data. In the embodiment described in FIG. 7, the viscoelasticity measurement performed on the target object is still based on different vibration signals, which can solve the problem of inaccurate and unstable measured result caused by using an ideal elasticity model, and improve the accuracy and stability of the measured result. In step S750, the elasticity parameter(s) or the viscosity parameter(s) may only be calculated, or both of them may be calculated and only one of them may be displayed. The different vibration signals may be generated based on different drive signals. Exemplarily, at least one of the following parameters of each drive signal for the different vibration signals is different: frequency, amplitude, phase and the number of periods. Exemplarily, the different vibration signals have different vibration waveforms from one another. Exemplarily, the different vibration waveforms differ in frequency from one another.

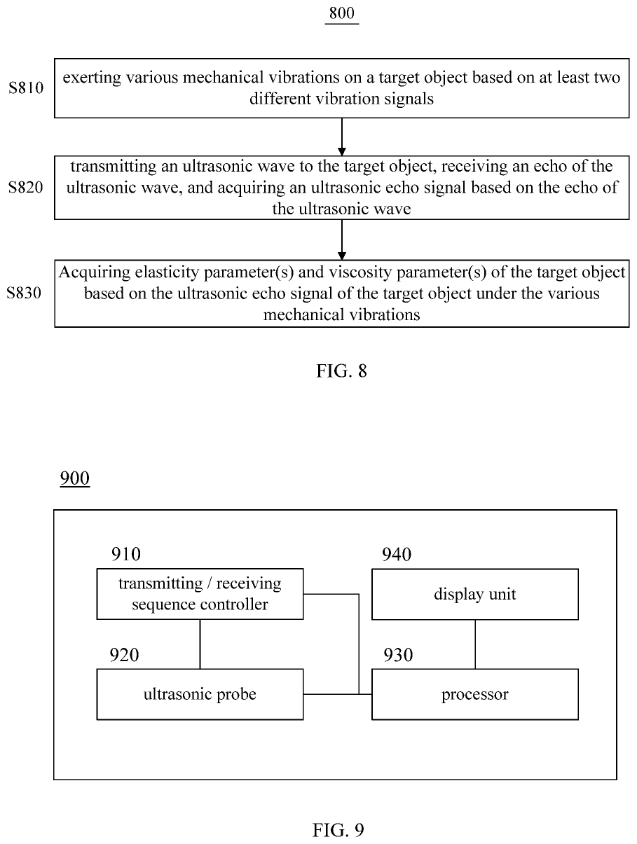

The ultrasonic viscoelasticity measuring method according to another embodiment of the present disclosure is described below with reference to FIG. 8. FIG. 8 shows a schematic flowchart of the ultrasonic viscoelasticity measuring method 800 according to another embodiment of the present disclosure. As shown in FIG. 8, the ultrasonic viscoelasticity measuring method 800 may include the following steps:

Step S810: exerting various mechanical vibrations on a target object based on at least two different vibration signals;

Step S820: transmitting an ultrasonic wave to the target object, receiving an echo of the ultrasonic wave, and acquiring an ultrasonic echo signal based on the echo of the ultrasonic wave; and

Step S830: acquiring elasticity parameter(s) and viscosity parameter(s) of the target object based on the ultrasonic echo signal of the target object under the various mechanical vibrations.

The core idea of the ultrasonic viscoelasticity measuring method 800 described with reference to FIG. 8 according to another embodiment of the present disclosure is similar to that of the ultrasonic viscoelasticity measuring method 500 described with reference to FIG. 5 according to the embodiment of the present disclosure, both of which relate to the ultrasonic viscoelasticity measurement of the target object based on different vibration signals. In the embodiment described with reference to FIG. 8, the region of interest of the target object can be acquired by any suitable means to perform the above viscoelasticity measurement without limiting the way in which it is acquired.

Exemplarily, the different vibration signals mentioned in step S810 may be generated based on different drive signals, and at least one of the following parameters of the different drive signals may be different: frequency, amplitude, phase and the number of periods. Exemplarily, the different vibration signals may have different vibration waveforms from one another. Exemplarily, the different vibration waveforms may differ in frequency from one another.

In an example, the target object may be performed with one measurement, in the measurement, mechanical vibrations may be exerted on the target object based on a plurality of different vibration signals, each vibration signal may correspond to one ultrasonic echo signal; and the elasticity parameter(s) and the viscosity parameter(s) of the target object may be acquired by calculating a group of elasticity parameter and viscosity parameter based on a plurality of ultrasonic echo signals corresponding to the plurality of different vibration signals. In an embodiment of the present disclosure, “one measurement” may, from clinical operation, be defined as being measured by a user by pressing a button once or inputting an instruction once or other operation once. Based on this, in this example, the user may obtain a group of elasticity parameter and viscosity parameter only with simple operation.

In another example, the target object may be performed with one measurement comprising multiple groups of sub-measurements, wherein in each group of sub-measurement, mechanical vibrations are exerted on the target object based on a plurality of different vibration signals, each vibration signal corresponds to one ultrasonic echo signal; and the elasticity parameter(s) and the viscosity parameter(s) of the target object may be acquired by: calculating the elasticity parameter(s) and the viscosity parameter(s) based on multiple groups of measured elasticity values and measured viscosity values, each group of measured elasticity value and measured viscosity value being calculated based on a plurality of ultrasonic echo signals corresponding to the plurality of different vibration signals in each group of sub-measurement; or, calculating to acquire multiple groups of elasticity parameters and viscosity parameters based on a plurality of ultrasonic echo signals corresponding to the plurality of different vibration signals in each group of sub-measurement. In this example, the user may still only need to press the button once or input the instruction once in other ways; however, unlike the previous example, this measurement includes multiple groups of sub-measurements, the multiple groups of measured elasticity values and the multiple groups of measured viscosity values obtained based on the multiple sub-measurement may be directly taken as the measured result of the viscoelasticity, and the measured result of the multiple groups of elasticity parameters and viscosity parameters may be obtained; and when further calculating the measured result of the viscoelasticity based on the multiple groups of measured elasticity values and measured viscosity values obtained in the multiple groups of sub-measurements, the elasticity parameter(s) and the viscosity parameter(s) may be improved.

Exemplarily, the multiple groups of sub-measurements are performed continuously in one measurement. Exemplarily, the target object is exerted with the same number of mechanical vibrations in each group of sub-measurement. Exemplarily, a group of different vibration signals is generated based on the same drive signal in each group of sub-measurement.

In still another example, the target object may be performed with a plurality of measurements, wherein in each of the plurality of measurements, mechanical vibrations may be exerted on the target object based on a plurality of different vibration signals, each vibration signal corresponds to one ultrasonic echo signal; and the elasticity parameter(s) and the viscosity parameter(s) of the region of interest may be acquired by calculating out multiple groups of elasticity parameters and viscosity parameters based on a plurality of ultrasonic echo signals corresponding to the multiple different vibration signals in each measurement. That is, a group of measured result of the elasticity parameter(s) and the viscosity parameter(s) may be outputted in each measurement. In this example, “a plurality of measurements” may, from clinical operation, be defined as being measured by a user by pressing a button for multiple times or inputting an instruction for multiple times or other operations for multiple times. Based on this, in this example, the user may obtain multiple groups of the measured elasticity values and the measured viscosity values after multiple operations and finally obtain multiple groups of elasticity parameters and viscosity parameters based on the multiple groups of measured elasticity values and measured viscosity values.

In yet still another example, the target object may be performed with a plurality of measurements, wherein in each of the plurality of measurements, mechanical vibrations may be exerted on the target object based on a plurality of different vibration signals, each vibration signal corresponds to one ultrasonic echo signal; and the elasticity parameter(s) and the viscosity parameter(s) of the region of interest may be acquired by calculating the elasticity parameter(s) and the viscosity parameter(s) based on multiple groups of measured elasticity values and measured viscosity values, where each group of measured elasticity value and measured viscosity value is calculated based on a plurality of ultrasonic echo signals acquired in each measurement. In this example, “a plurality of measurements” may, from clinical operation, be defined as being measured by a user by pressing a button for multiple times or inputting an instruction for multiple times or other operations. Based on this, in this example, the user may obtain multiple groups of the measured elasticity values and the measured viscosity values after multiple operations and finally obtain multiple groups of elasticity parameters and viscosity parameters based on the multiple groups of the measured elasticity values and the measured viscosity values.

Exemplarily, during performing the plurality of measurements on the target object, the number and/or waveforms of the vibration signals used in each measurement may be different. Exemplarily, the elasticity parameter may be equal to a weighted average of part or all of multiple measured elasticity values, or equal to one of the multiple measured elasticity values; and the viscosity parameter is equal to a weighted average of part or all of multiple measured viscosity values, or equal to one of the multiple measured viscosity values.

Exemplarily, during each measurement performed on the target object, at least one of the following parameters of each drive signal for the plurality of different vibration signals may be different: frequency, amplitude, phase and the number of periods, and at least one of the following parameters of the different vibration signals may be different: frequency, amplitude, phase and the number of periods.

Exemplarily, the target object may be performed with each measurement based on a received instruction inputted by a user that at least includes viscoelasticity measurement, or based on other preset conditions. Exemplarily, in each measurement, after mechanical vibrations may be exerted on the target object based on one vibration signal to obtain a corresponding ultrasonic echo signal, the target object may be exerted with mechanical vibrations based on another vibration signal after a predetermined cooling time.

Exemplarily, in the embodiment of the present disclosure, at least one of the elasticity parameter and the viscosity parameter may be displayed; or the multiple groups of measured elasticity values and measured viscosity values together with the elasticity parameter(s) and the viscosity parameter(s) may be displayed. Exemplarily, in the embodiment of the present disclosure, the ultrasonic echo signal may also be generated based on step S820 and the ultrasonic image may be displayed.

The above exemplifies the ultrasonic viscoelasticity measuring methods according to the embodiments of the present disclosure. In general, with these methods, ultrasonic viscoelasticity measurement may be performed on a target object on the basis of external vibration under different excitation, which can obtain elasticity parameter(s) and viscous parameter(s) of a region of interest of the target object, solving the problem of inaccurate and unstable measurement when using the ideal elasticity model, and improving the accuracy and stability of the measurement.

An ultrasonic viscoelasticity measuring apparatus according to the embodiment of the present application is described below with reference to FIGS. 9-13, which can be used to implement the ultrasonic viscoelasticity measuring methods according to the embodiments of the present invention described above.

A schematic block diagram of an ultrasonic viscoelasticity measuring apparatus 900 in an embodiment of the present disclosure is described below with reference to FIG. 9. As shown in FIG. 9, the ultrasonic viscoelasticity measuring apparatus 900 may include a transmitting/receiving sequence controller 910, an ultrasonic probe 920, a processor 930 and a display unit 940. The ultrasonic viscoelasticity measuring apparatus 900 according to an embodiment of the present disclosure may be configured for performing the ultrasonic viscoelasticity measuring methods 500/600/700 according to the embodiments of the present disclosure described above.

Specifically, the ultrasonic probe 920 may include a vibrator and a transducer (not shown). The vibrator may be configured to drive the transducer to vibrate to generate a shear wave propagating in a depth direction inside a target object. The transducer may include a plurality array elements; at least part of the array elements may be configured to, before the transducer is vibrated, transmit a first ultrasonic wave to a target object, receive an echo of the first ultrasonic wave, and acquire a first ultrasonic echo signal based on the echo of the ultrasonic wave; and after the transducer is vibrated, transmit a second ultrasonic wave to a region of interest of the target object, receive an echo of the second ultrasonic wave, and acquire a second ultrasonic echo signal based on the echo of the second ultrasonic wave. The transmitting/receiving sequence controller 910 may be configured to, before the transducer is vibrated, output a first transmitting/receiving sequence to the transducer to control the transducer to transmit the first ultrasonic wave, receive the echo of the first ultrasonic wave and acquire the first ultrasonic echo signal based on the echo of the first ultrasonic wave; and after the region of interest is determined, output different drive signals to control the vibrator to drive the transducer to exert various mechanical vibrations on the target object based on at least two different vibration signals, and at least after the transducer is vibrated, output a second transmitting/receiving sequence to the transducer to control the transducer to transmit the second ultrasonic wave, receive the echo of the second ultrasonic wave and acquire the second ultrasonic echo signal based on the echo of the second ultrasonic wave. The processor 930 may be configured to generate an ultrasonic image based on the first ultrasonic echo signal, acquire a region of interest of the ultrasonic image, and acquire elasticity parameter(s) and viscosity parameter(s) of the region of interest based on the second ultrasonic echo signal of the region of interest under various mechanical vibrations. The display unit 940 may be configured to display the elasticity parameter(s) and the viscosity parameter(s) of the region of interest.

In the embodiment of the present disclosure, the vibrator of the ultrasonic probe 920 is installed on the ultrasonic probe 920 (for example, installed on a housing of the ultrasonic probe 920, or installed in the housing of the ultrasonic probe 920), and assembled with the transducer and other probe components into an integrated ultrasonic probe. The transmit/receive sequence controller 910 may output a drive signal to control the vibrator which per se can vibrate according to a vibration sequence and drive the transducer to vibrate; or the vibrator itself does not vibrate, but drives the transducer to vibrate through a telescopic component. Such vibration may lead to deformation of the target object when the ultrasonic probe 920 contacts the target object, generating a shear wave propagating in the depth direction inside the internal target object.

In the embodiment of the present disclosure, the transducer of the ultrasonic probe 920 may include a plurality of array elements arranged in an array. A plurality of array elements are arranged in a row to form a linear array; or arranged into a two-dimensional matrix to form a plane array. The plurality of array elements may also form a convex array. The array elements may be used to transmit an ultrasonic wave according to excitation of an electrical signal, or convert the received ultrasonic wave into the electrical signal. Therefore, each array element may be used to transmit the ultrasonic wave to a biological tissue in the region of interest, and may also be used to receive an ultrasonic echo returned from the tissue. During ultrasonic testing, the transmit/receive sequence controller 910 may control which array elements are used to transmit the ultrasonic wave and which array elements are used to receive the ultrasonic wave, or control the array elements to transmit or receive the ultrasonic wave in time slots. The array elements participating in ultrasonic transmission can be excited by electrical signals at the same time, so as to transmit the ultrasonic wave simultaneously; or the array elements participating in ultrasonic beam emission can also be excited by several electrical signals with a certain time interval, so as to continuously emit ultrasonic waves with a certain time interval.

In the embodiment of the present disclosure, the transmitting/receiving sequence controller 910 may be used to generate a transmitting sequence and a receive sequence. The transmitting sequence may be used to control part or all of the array elements to transmit the ultrasonic wave to the target object. Transmitting sequence parameters may include the position of the array elements used for transmitting, the number of array elements, and ultrasonic transmitting parameters (such as amplitude, frequency, frequency of transmitting wave, transmitting interval, transmitting angle, waveform, focusing position, etc.). The receiving sequence may be used to control part or all of the multiple array elements to receive echo received from the tissue. Receiving sequence parameters may include the position of array elements used for receiving, number of array elements, and receiving parameters of echo (such as receiving angle and depth, etc.). The ultrasonic parameters in the transmitting sequence and the echo parameters in the receiving sequence may be different with different uses of the ultrasonic echo, different images generated by the ultrasonic echo, and different detection types.

In an embodiment of the present disclosure, the transmitting/receiving sequence outputted to the transducer of the ultrasonic probe 920 by the transmitting/receiving sequence controller 910 may include a first transmitting/receiving sequence and a second transmitting/receiving sequence. The first transmitting/receiving sequence may be for the purpose of obtaining an ultrasonic image, that is, the ultrasonic transmitting parameter and receiving parameter may be determined according to the requirements of generating ultrasonic images. The first transmitting/receiving sequence may be outputted before the vibration of the transducer or after the vibration of the transducer to control the transducer to transmit the first ultrasonic wave and receive the echo of the first ultrasonic wave. The second transmitting/receiving sequence may aim to detect the viscoelasticity of the region of interest, that is, the ultrasonic transmitting parameter and receiving parameter are determined according to the requirements of detecting the transient viscoelasticity of the region of interest, for example, the ultrasonic transmitting angle, receiving angle and depth, transmission radio frequency rate and other parameters may be determined according to the region of interest. The transmitting/receiving sequence controller 910 may output a second transmitting/receiving sequence to the transducer after the transducer is vibrated, which may be used to control the transducer to transmit the second ultrasonic wave and receive the echo of the second ultrasonic wave.

Further, in the embodiment of the present disclosure, the ultrasonic viscoelasticity measuring apparatus 900 may also include a transmitting circuit and a receiving circuit (not shown), which may be coupled between the ultrasonic probe 920 and the transmitting/receiving sequence controller 910 to transmit the transmitting/receiving sequence outputted by the transmitting/receiving sequence controller 910 to the ultrasonic probe 920. In addition, the ultrasonic viscoelasticity measuring apparatus 900 may also include an echo processing unit (not shown), and the receiving circuit may also be used to transmit the ultrasonic echo received by the ultrasonic probe 920 to the echo processing unit. The echo processing unit may be used to process the ultrasonic echo, such as performing filtering, amplification, beam synthesis, etc. on the ultrasonic echo. The ultrasonic echo in the embodiment of the present disclosure may include the echo of the second ultrasonic wave used for detecting the transient viscoelasticity, and also the echo of the first ultrasonic wave used for generating the ultrasonic image. The ultrasonic image may be, for example, a B image or a C image, or a combination of both. The echo processing unit may also be included in the processor 930.

In the embodiment of the present disclosure, the processor 930 may obtain a required parameter or image using a corresponding algorithm based on the echo signal processed by the echo processing unit or the ultrasonic echo signal acquired based on the ultrasonic probe 920. In the embodiment of the present disclosure, the processor 930 may process the first ultrasonic echo signal to generate ultrasonic image data. In addition, the processor 930 may process the second ultrasonic echo signal to calculate the viscoelasticity of the region of interest.

In an embodiment of the present disclosure, the vibrator may be driven to vibrate by using different drive signals, thereby implementing viscoelasticity measurement. Different drive signals outputted by the vibrator make the vibrator to exert different mechanical vibrations on the target object based on at least two different vibration signals. Exemplarily, the difference among the vibration signals may be shown as follows: different vibration signals being different from each other in vibration waveform; different vibration signals being different from each other in different frequencies; or any other possible difference. Using different drive signals to drive the vibrator to perform viscoelasticity measurement may enable the vibrator to exert different mechanical vibrations under different vibration signals, thus obtaining a shear wave data of the region of interest of the target object under different mechanical vibrations. Further, based on the shear wave data of the region of interest of the target object under different mechanical vibrations, a more stable and accurate measured result about elasticity and viscosity can be obtained.

In an example, the processor 930 may control to perform the target object with one measurement. Such measurement may be implemented by exerting mechanical vibrations on the target object based on different vibration signals, wherein each vibration signal corresponds to one ultrasonic echo signal; and the elasticity parameter(s) and the viscosity parameter(s) of the region of interest may be acquired by calculating a group of measured elasticity value and measured viscosity value based on a plurality of ultrasonic echo signals corresponding to the plurality of different vibration signals, thereby acquiring the elasticity parameter and the viscosity parameter respectively based on the group of measured elasticity value and measured viscosity value. In an embodiment of the present disclosure, “one measurement” may, from clinical operation, be defined as being measured by a user by pressing a button once or inputting an instruction once or other operation once. In this respect, in this example, the user may obtain a group of elasticity parameter and viscosity parameter only with simple operation.

In another example, the processor 930 may perform on the target object with one measurement that includes multiple groups of sub-measurements, wherein in each group of sub-measurement, the target object may be exerted with mechanical vibrations based on a plurality of different vibration signals, each vibration signal corresponds to one ultrasonic echo signals; and the elasticity parameter(s) and the viscosity parameter(s) of the region of interest may be acquired by calculating to acquire multiple groups of elasticity parameters and viscosity parameters based on the plurality of ultrasonic echo signals corresponding to the plurality of different vibration signals in each group of sub-measurement. In this example, the user may still only need to press the button once or input the instruction once in other ways; however, unlike the aforesaid example, this measurement includes multiple groups of sub-measurements, and multiple groups of measured elasticity values and multiple groups of measured viscosity values obtained directly based on the multiple groups of sub-measurements may be taken as the measured result of viscoelasticity, thus the multiple groups of elasticity parameters and viscosity parameters can be measured.

In yet another example, the processor 930 may perform on the target object with one measurement that includes multiple groups of sub-measurements, wherein in each group of sub-measurement, the target object may be exerted with mechanical vibrations based on a plurality of different vibration signals, each vibration signal corresponds to one ultrasonic echo signals; and the elasticity parameter(s) and the viscosity parameter(s) of the region of interest may be acquired by calculating the elasticity parameter(s) and the viscosity parameter(s) based on multiple groups of measured elasticity values and measured viscosity values, where each group of measured elasticity value and measured viscosity value is calculated based on a plurality of ultrasonic echo signals corresponding to the plurality of different vibration signals in each group of sub-measurement. In this example, the user may still only need to press the button once or input the instruction once in other ways; however, unlike the previous example, this measurement includes multiple groups of sub-measurements, and the result of the viscoelasticity in this example is further calculated based on the multiple groups of the measured elasticity value and the measured viscosity value, thereby acquiring a more accurate measured result of the elasticity parameter(s) and the viscosity parameter(s).

Exemplarily, the multiple groups of sub-measurements may be the ones that perform measuring continuously in one measurement. Said measuring continuously may refer to that after a previous group of sub-measurement is completed, a next group of sub-measurement may be started automatically after a predetermined time interval without a startup command inputted again by the user between the two groups of sub-measurements. Exemplarily, the target object may be exerted with the same number of mechanical vibrations in each group of sub-measurement. Exemplarily, a group of different vibration signals may be generated based on the same drive signal in each group of sub-measurement. For each group of sub-measurement, with exerting mechanical vibrations of the same number to the target object, and/or with generation of a group of different vibration signals based on the same drive signal, each group of sub-measurement can be performed under the same external conditions, thereby obtaining a more accurate measured result.

In other examples, during performing a plurality of sub-measurement on the target object, the number and/or waveforms of the vibration signals used in each sub-measurement may be different. Exemplarily, during each measurement performed on the target object, at least one of the following parameters of each drive signal for the plurality of different vibration signals is different: frequency, amplitude, phase and the number of periods, and at least one of the following parameters of the different vibration signals is different: frequency, amplitude, phase and the number of periods. In general, the drive signals and actual vibration waveform may be unequal, and there may be a differential relationship therebetween in the ideal model.

In still another example, the processor 930 may perform on the target object with a plurality of measurements, wherein in each of the plurality of measurements, mechanical vibrations may be exerted on the target object based on a plurality of different vibration signals, each vibration signal corresponds to one ultrasonic echo signal; and the elasticity parameter(s) and the viscosity parameter(s) of the region of interest may be acquired by calculating multiple groups of elasticity parameters and viscosity parameters based on a plurality of ultrasonic echo signals corresponding to the multiple different vibration signals in each measurement. That is, a group of measured result of the elasticity parameter(s) and the viscosity parameter(s) may be outputted in each measurement. In this example, “a plurality of measurements” may, from clinical operation, be defined as being measured by a user by pressing a button for multiple times or inputting an instruction for multiple times or other operations for multiple times. Based on this, in this example, the user may obtain multiple groups of measured elasticity values and measured viscosity values after multiple operations and finally obtain multiple groups of elasticity parameters and viscosity parameters based on the multiple groups of measured elasticity values and measured viscosity values.

In yet still another example, the target object may be performed with a plurality of measurements, wherein in each of the plurality of measurements, mechanical vibrations may be exerted on the target object based on a plurality of different vibration signals, each vibration signal corresponds to one ultrasonic echo signal; and the elasticity parameter(s) and the viscosity parameter(s) of the region of interest may be acquired by calculating the elasticity parameter(s) and the viscosity parameter(s) based on multiple groups of measured elasticity values and measured viscosity values, where each group of the measured elasticity value and the measured viscosity value is calculated based on a plurality of ultrasonic echo signals acquired in each measurement. In this example, “a plurality of measurements” may, from clinical operation, be defined as being measured by a user by pressing a button for multiple times or inputting an instruction for multiple times or other operations. Based on this, in this example, the user may obtain multiple groups of the measured elasticity values and the measured viscosity values after multiple operations and finally obtain multiple groups of elasticity parameters and viscosity parameters based on the multiple groups of measured elasticity values and measured viscosity values.

Exemplarily, during performing the plurality of measurements on the target object, the number and/or waveforms of the vibration signals used in each measurement may be different. Exemplarily, during each measurement performed on the target object, at least one of the following parameters of each drive signal for the plurality of different vibration signals may be different: frequency, amplitude, phase and the number of periods; and at least one of the following parameters of the different vibration signals may be different: frequency, amplitude, phase and the number of periods. In general, the drive signal and actual vibration waveform may be unequal, and there may be a differential relationship therebetween in the ideal model.

In an embodiment of the present disclosure, the processor 930 may perform each measurement on the target object based on a received instruction inputted by a user that at least includes viscoelasticity measurement, or based on other preset conditions. In addition, for example, in each measurement, after mechanical vibrations may be exerted on the target object based on one vibration signal to obtain a corresponding ultrasonic echo signal, the target object may be exerted with mechanical vibrations based on another vibration signal after a predetermined cooling time, thereby acquiring a more accurate measured result.

In an embodiment of the present disclosure, the display unit 940 may display the ultrasonic image based on the ultrasonic image data generated by the processor 930. The region of interest of the target object on the ultrasonic image may be manually selected by the user via an input unit (not shown). Alternatively, the processor 930 may automatically detect the region of interest of the target object on the ultrasonic image based on related algorithms. Alternatively, a rough region may be selected first by the user, and then a more accurate region within the rough region selected by the user may be automatically detected based on a certain algorithm by the processor 930 to obtain the region of interest; or, the region of interest on the ultrasonic image may be first automatically detected by the processor 930 based on a certain algorithm, and then be modified or corrected by the user to obtain a more accurate region of interest.

In another embodiment of the present disclosure, the acquired measured results of elasticity and viscosity may be displayed by the display unit 940. Exemplarily, via the display unit 940, each group of measured elasticity value and measured viscosity value may be displayed, or only the measured results of elasticity and viscosity that are calculated respectively based on the measured elasticity values and the measured viscosity values may be displayed. Further, the ultrasonic image may be displayed by the display unit 940 while displaying the elasticity parameter(s) and the viscosity parameter(s) of the region of interest. The ultrasonic image may be generated based on the first ultrasonic echo signal or the second ultrasonic echo signal. For example, the elasticity parameter(s)/measured value(s) and the viscosity parameter(s)/measured value(s) of the region of interest may be displayed by the display unit 940 at an appropriate location in the ultrasonic image (e.g. at lower right corner or at middle of region of interest), or may be displayed in a non-image region, such as they may be side by side with the ultrasonic image.

The above example shows the ultrasonic viscoelasticity measuring apparatus 900 according to an embodiment of the present disclosure. Based on the above description, the ultrasonic viscoelasticity measuring apparatus 900 according to an embodiment of the present disclosure may perform ultrasonic viscoelasticity measurement on the target object based on external vibrations under different excitation, which can obtain the elasticity parameter(s) and the viscoelasticity parameter(s) of the region of interest of the target object, solving the problem of inaccurate and unstable measured result caused when using an ideal elasticity model, and improving the accuracy and stability of the measured result.

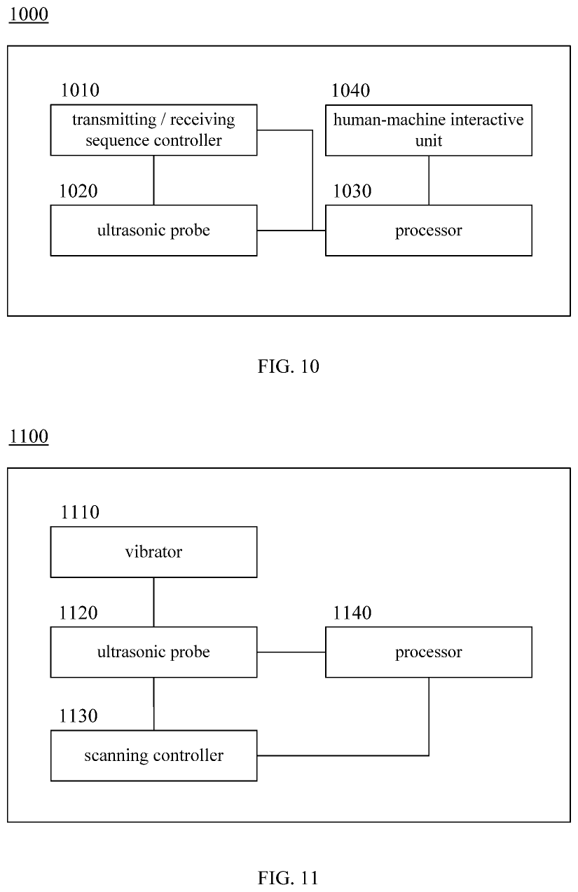

A schematic block diagram of an ultrasonic viscoelasticity measuring apparatus 1000 in another embodiment of the present disclosure is described below with reference to FIG. 10. As shown in FIG. 10, the ultrasonic viscoelasticity measuring apparatus 1000 may include a transmitting/receiving sequence controller 1010, an ultrasonic probe 1020, a processor 1030 and a human-machine interactive unit 1040. The ultrasonic viscoelasticity measuring apparatus 1000 according to an embodiment of the present disclosure may be configured for performing the ultrasonic viscoelasticity measuring method 700 according to the embodiment of the present disclosure described above.

Specifically, the ultrasonic probe 1020 may include a vibrator and a transducer (not shown). The vibrator may be configured to drive the transducer to vibrate to generate a shear wave propagating in the depth direction inside a target object. The transducer may include one or more array elements; at least part of the array elements may be configured to, after the transducer is vibrated, transmit an ultrasonic wave to a region of interest of a target object, receive an echo of the ultrasonic wave, and acquire an ultrasonic echo signal based on the echo of the ultrasonic wave. The transmitting/receiving sequence controller 1010 may be configured to, after the region of interest is determined, output different drive signals to control the vibrator to drive the transducer to exert various mechanical vibrations on the target object based on at least two different vibration signals, and at least after the transducer is vibrated, output a transmitting/receiving sequence to the transducer to control the transducer to transmit the ultrasonic wave, receive the echo of the ultrasonic wave and acquire the ultrasonic echo signal based on the echo of the ultrasonic wave. The processor 1030 may be configured to acquire a tissue image of the target object, acquire a region of interest of the tissue image, and acquire elasticity parameter(s) and viscosity parameter(s) of the region of interest based on the ultrasonic echo signal of the region of interest under various mechanical vibrations. The human-machine interactive unit 1040 may be configured to detect the region of interest selected on the tissue image by a user, and display the elasticity parameter(s) and the viscosity parameter(s) of the region of interest.

There are only slight difference between the ultrasonic viscoelasticity measuring apparatus 1000 described with reference to FIG. 10 according to another embodiment of the present disclosure and the ultrasonic viscoelasticity measuring apparatus 900 described with reference to FIG. 9 according to the embodiment of the present disclosure. For simplicity, the same details will not be repeated here. In the embodiment described with reference to FIG. 10, the tissue image of the target object may be acquired in real time or from a storage medium. In addition, in the embodiment described with reference to FIG. 10, the region of interest may be selected on the tissue image by the user via the human-machine interactive unit 1040 so as to generate the shear wave within the region of interest. The human-machine interactive unit 1040 may not be an essential component; instead, the region of interest may be determined on the tissue image through automatic image recognition and other methods.

In an embodiment, the human-machine interactive unit 1040 may include a display and an input unit. The input unit may for example be a keyboard, an operation button, a mouse, a trackball and the like, or be a touch screen integrated with the display. In a case where the input unit is a keyboard or an operation button, a user may input operation information or operation instruction(s) via the input unit. In a case where the input unit is a mouse, a trackball or a touch screen, the user may input the operation information or the operation instruction(s) via the input unit in combination with a soft button, an operation icon, a menu option, etc. on the display interface, or may input the operation information by marking, framing, etc. on the display interface. The operation instruction(s) may be an instruction for entering an ultrasonic image measurement mode, an instruction for entering a viscoelasticity measurement mode, or an instruction for entering a simultaneous measurement mode of viscoelasticity and ultrasonic image. In an embodiment, the selection of the region of interest may be realized by combining the display with the input unit. For example, the display is configured to display the ultrasonic image on the display interface, and the input unit is configured to select the region of interest on the ultrasonic image in accordance with the user's operation.

In addition, the display may also be configured to display the measured result of viscoelasticity. For example, both the ultrasonic image and the measured result of viscoelasticity are displayed in the display interface, or only the measured result of viscoelasticity is shown after it is measured without displaying the ultrasonic image. When displaying the measured result of viscoelasticity, only the viscosity parameter(s) or the elasticity parameter(s) may be displayed, or both of them may be displayed simultaneously.

In the embodiment described with reference to FIG. 10, the viscoelasticity measurement performed on the target object is still based on different vibration signals, which can solve the problem of inaccurate and unstable measured result caused when using an ideal elasticity model, and improve the accuracy and stability of the measured result.

A schematic block diagram of an ultrasonic viscoelasticity measuring apparatus 1100 in another embodiment of the present disclosure is described below with reference to FIG. 11. As shown in FIG. 11, the ultrasonic viscoelasticity measuring apparatus 1100 may include a vibrator 1110, an ultrasonic probe 1120, a scanning controller 1130 and a processor 1140. The ultrasonic viscoelasticity measuring apparatus 1100 according to an embodiment of the present disclosure may be configured for performing the ultrasonic viscoelasticity measuring method 800 according to the embodiment of the present disclosure described above.

Specifically, the vibrator 1110 may be configured to exert various mechanical vibrations on a target object based on at least two different vibration signals. The scanning controller 1130 may be configured to excite the ultrasonic probe 1120 to transmit an ultrasonic wave to the target object, receive an echo of the ultrasonic wave, and acquire an ultrasonic echo signal based on the echo of the ultrasonic wave. The processor 1140 may be configured to acquire the elasticity parameter(s) and the viscosity parameter(s) of the target object based on the ultrasonic echo signal of the target object under various mechanical vibrations.

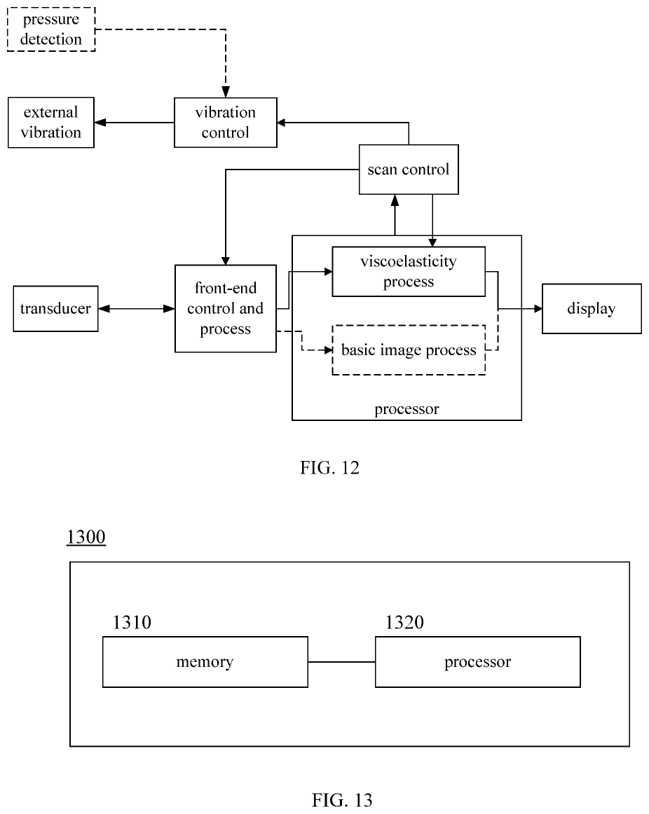

In the embodiment described with reference to FIG. 11, the vibration signals of the vibrator 1110 may be generated according to different drive signals which may be generated by a vibration controller (not shown) or the scanning controller 1130. Further, the ultrasonic viscoelasticity measuring apparatus 1100 may also include a pressure sensor (not shown) whose output is coupled to the scanning controller 1130 for feeding back a sensed pressure and a sensed vibration intensity that the vibrator exerts on the target object to the scanning controller 1130. Further, the scanning controller 1130 may also be configured to control the vibrator 1110 to vibrate when the value of the pressure is within a preset range. Exemplarily, the viscoelasticity measurement performed by the ultrasonic viscoelasticity measuring apparatus 1100 can be understood in combination with FIG. 12.

In the embodiment described with reference to FIG. 11, the viscoelasticity measurement performed on the target object is still based on different vibration signals, which can solve the problem of inaccurate and unstable measured result caused by using an ideal elasticity model, and improve the accuracy and stability of the measured result.

FIG. 12 depicts a schematic block diagram of an ultrasonic viscoelasticity measuring apparatus according to another embodiment of the present disclosure. The ultrasonic viscoelasticity measuring apparatus may include an ultrasonic probe, a front-end control and processing unit, a processor, a scanning controller and a display. The ultrasonic viscoelasticity measuring apparatus according to an embodiment of the present disclosure may be used to perform the ultrasonic viscoelasticity measuring methods 500, 700, 800 according to the embodiments of the present disclosure described above.

The ultrasonic probe may include a transducer and a vibrator. Under the control of the scanning controller, the transducer of the ultrasonic probe may transmit an ultrasonic wave to a target object, receive an echo of the ultrasonic wave, and acquire an ultrasonic echo signal based on the ultrasonic echo. The vibrator may be configured to, under the control of the scanning controller, exert various mechanical vibrations on the target object based on at least two different vibration signals, thereby generating a shear wave within a region of interest of the target object. The scanning controller may include a transmitting/receiving sequence controller, through which a transmitting/receiving sequence is outputted to control the transducer to perform ultrasonic scanning, a drive signal is outputted to control the vibrator to exert mechanical vibration. For the specific description of the transmitting/receiving sequence controller, please refer to the previous description, which will not be repeated here.

The front-end control and processing unit may include a filtering circuit, an amplification circuit, an analog-to-digital conversion circuit, a beam synthesis unit and the like, which are used to perform filtering, amplifying, beam forming and other processes on the ultrasonic echo signal acquired by the ultrasonic probe. The ultrasonic echo signal after beam synthesis is sent to the processor which may process the beam formed ultrasonic echo signal according to different imaging modes. For example, it may process the beam formed ultrasonic echo signal to obtain a B image, a C image or an M image, etc. The processor may also process the beam formed ultrasonic echo signal under various mechanical vibrations to obtain viscosity parameter(s) and/or elasticity parameter(s) of the region of interest.

The ultrasonic probe may further provide with a pressure sensor to detect the pressure between the ultrasonic probe and the target object. The pressure may include an initial pressure before the measurement and a pressure during the measurement; and the processor may judge the validity of the measured result of viscoelasticity according to a pressure signal outputted by the pressure sensor. Specifically, the processor may judge the validity of the measured result of viscoelasticity according to whether the pressure signal falls into a preset pressure range. A schematic block diagram of an ultrasonic viscoelasticity measuring apparatus according to another embodiment of the present disclosure is described below with reference to FIG. 13. FIG. 13 shows a schematic block diagram of an ultrasonic viscoelasticity measuring apparatus 1300 according to an embodiment of the present disclosure. The ultrasonic viscoelasticity measuring apparatus 1300 may include a memory 1310 and a processor 1320.

The memory 1310 may store a program for realizing the corresponding steps in the ultrasonic viscoelasticity measuring methods 500, 700, 800 according to the embodiments of the present disclosure. The processor 1320 may be configured to run the program stored in the memory 1310 to execute the corresponding steps in the ultrasonic viscoelasticity measuring methods 500, 700, 800 according to the embodiments of the present disclosure.

In addition, according to an embodiment of the present disclosure, there is provided a storage medium, on which program instructions are stored, and the program instructions are run by a computer or a processor, the corresponding steps of the ultrasonic viscoelasticity measuring methods 500, 700, 800 of the embodiments of the present disclosure are executed. The storage medium may include, for example, a memory card of a smart phone, a storage component of a tablet computer, a hard disk of a personal computer, a read-only memory (ROM), an erasable programmable read-only memory (EPROM), a portable compact disk read-only memory (CD-ROM), a USB memory, or any combination of the above storage media. The computer-readable storage medium may be any combination of one or more computer-readable storage media.

In addition, according to an embodiment of the present disclosure, there is also provided a computer program, which can be stored on the cloud or local storage medium. When the computer program is run by a computer or processor, it is used to perform the corresponding steps of the ultrasonic viscoelasticity measuring method of the embodiment of the present disclosure.

Based on the above description, with the ultrasonic viscoelasticity measuring method, apparatus and storage medium according to embodiments of the present disclosure, ultrasonic viscoelasticity measurement may be performed on a target object on the basis of external vibration with different excitation, which can obtain elasticity parameter(s) and viscous parameter(s) of a region of interest of the target object, solving the problem of inaccurate and unstable measurement when using the ideal elasticity model, and improving the accuracy and stability of the measurement.

While exemplary embodiments have been described herein with reference to the accompanying drawings, it should be understood that the above example embodiments are merely illustrative and are not intended to limit the scope of the disclosure thereto. Those skilled in the art may make various changes and modifications therein without departing from the scope and spirit of the disclosure. All such changes and modifications are intended to be included in the scope of the disclosure as claimed in the appended claims.

A person of ordinary skill in the art may be aware that, in combination with the examples described in the embodiments disclosed in this specification, units and algorithm steps may be implemented by using electronic hardware or a combination of computer software and electronic hardware. Whether the functions are performed by hardware or software depends on particular applications and design constraint conditions of the technical solutions. Those skilled in the art could use different methods to implement the described functions for each particular application, but such implementation should not be considered to be beyond the scope of the disclosure.

In several embodiments provided in the present disclosure, it should be understood that the disclosed devices and methods may be implemented in other ways. For example, the device embodiments described above are merely exemplary. For example, the division of units is merely a logical function division. In actual implementations, there may be other division methods. For example, a plurality of units or components may be combined or integrated into another device, or some features may be omitted or not implemented.

A large number of specific details are explained in this specification provided herein. However, it can be understood that the embodiments of the disclosure can be practiced without these specific details. In some instances, well-known methods, structures, and technologies are not shown in detail, so as not to obscure the understanding of this description.

Similarly, it should be understood that in order to simplify the disclosure and help to understand one or more of various aspects of the disclosure, in the description of the exemplary embodiments of the disclosure, various features of the disclosure are sometimes together grouped into an individual embodiment, figure or description thereof. However, the method of the disclosure should not be construed as reflecting the following intention, namely, the disclosure set forth requires more features than those explicitly stated in each claim. More precisely, as reflected by the corresponding claims, the inventive point thereof lies in that features that are fewer than all the features of an individual embodiment disclosed may be used to solve the corresponding technical problem. Therefore, the claims in accordance with the particular embodiments are thereby explicitly incorporated into the particular embodiments, wherein each claim itself serves as an individual embodiment of the disclosure.

Those skilled in the art should understand that, in addition to the case where features are mutually exclusive, any combination may be used to combine all the features disclosed in this specification (along with the appended claims, abstract, and drawings) and all the processes or units of any of methods or devices as disclosed. Unless explicitly stated otherwise, each feature disclosed in this specification (along with the appended claims, abstract, and drawings) may be replaced by an alternative feature that provides the same, equivalent, or similar object.

Furthermore, those skilled in the art should understand that although some of the embodiments described herein comprise some but not other features included in other embodiments, combinations of features of different embodiments are meant to be within the scope of the disclosure, and form different embodiments. For example, in the claims, any one of the embodiments set forth thereby can be used in any combination.

Various embodiments regarding components in the disclosure may be implemented in hardware, or implemented by software modules running on one or more processors, or implemented in a combination thereof. It should be understood for those skilled in the art that a microprocessor or a digital signal processor (DSP) may be used in practice to implement some or all of the functions of some modules according to the embodiments of the disclosure. The disclosure may further be implemented as an apparatus program (e.g. a computer program and a computer program product) for executing some or all of the methods described herein. Such a program for implementing the disclosure may be stored on a computer-readable medium, or may be in the form of one or more signals. Such a signal may be downloaded from an Internet website, or provided on a carrier signal, or provided in any other form.

It should be noted that the description of the disclosure made in the above-mentioned embodiments is not to limit the disclosure, and those skilled in the art may design alternative embodiments without departing from the scope of the appended claims. In the claims, any reference signs placed between parentheses should not be construed as limitation on the claims. The word “comprising” does not exclude the presence of elements or steps not listed in a claim. The word “a” or “an” preceding an element does not exclude the presence of a plurality of such elements. The disclosure may be implemented by means of hardware comprising several different elements and by means of an appropriately programmed computer. In unit claims listing several ultrasound devices, several of these ultrasound devices may be specifically embodied by one and the same item of hardware. The use of the terms “first”, “second”, “third”, etc. does not indicate any order. These terms may be interpreted as names.

The above is only the specific embodiment of the present disclosure or the description of the specific embodiment, and the protection scope of the present disclosure is not limited thereto. Any changes or substitutions should be included within the protection scope of the present disclosure. The protection scope of the present disclosure shall be subject to the protection scope of the claims.

1. An ultrasonic viscoelasticity measuring method, comprising:
outputting a first transmitting/receiving sequence to a transducer of an ultrasonic probe to control the transducer to transmit a first ultrasonic wave to a target object, receive an echo of the first ultrasonic wave, and acquire a first ultrasonic echo signal based on the echo of the first ultrasonic wave;
generating an ultrasonic image based on the first ultrasonic echo signal and displaying the ultrasonic image, and acquiring a region of interest on the ultrasonic image;
outputting different drive signals to a vibrator of the ultrasonic probe to drive the transducer by the vibrator to exert various mechanical vibrations on the target object based on at least two different vibration signals;
outputting a second transmitting/receiving sequence to the transducer to control the transducer to transmit a second ultrasonic wave to the region of interest, receive an echo of the second ultrasonic wave, and acquire a second ultrasonic echo signal based on the echo of the second ultrasonic wave; and
acquiring and displaying elasticity parameter(s) and viscosity parameter(s) of the region of interest based on the second ultrasonic echo signal of the region of interest under the various mechanical vibrations.

2. The method according to claim 1, wherein the different vibration signals have different vibration waveforms from one another.

3. The method according to claim 2, wherein the different vibration waveforms differ in frequency from one another.

4. The method according to claim 1, further comprising performing on the target object with one measurement, wherein in said one measurement, mechanical vibrations are exerted on the target object based on a plurality of different vibration signals, each vibration signal corresponds to one ultrasonic echo signal; and
said acquiring elasticity parameter(s) and viscosity parameter(s) of the region of interest comprises calculating a group of elasticity parameter and viscosity parameter based on a plurality of ultrasonic echo signals corresponding to the plurality of different vibration signals.

5. The method according to claim 1, further comprising performing on the target object with one measurement comprising multiple groups of sub-measurements, wherein in each group of sub-measurement, mechanical vibrations are exerted on the target object based on a plurality of different vibration signals, each vibration signal corresponds to one ultrasonic echo signal; and
said acquiring elasticity parameter(s) and viscosity parameter(s) of the region of interest comprises:
calculating the elasticity parameter(s) and the viscosity parameter(s) based on multiple groups of measured elasticity values and measured viscosity values, each group of measured elasticity value and measured viscosity value being calculated based on a plurality of ultrasonic echo signals corresponding to the plurality of different vibration signals in each group of sub-measurement;
or, calculating to acquire multiple groups of elasticity parameters and viscosity parameters based on a plurality of ultrasonic echo signals corresponding to the plurality of different vibration signals in each group of sub-measurement.

6. The method according to claim 5, wherein the multiple groups of sub-measurements are performed continuously in said one measurement.

7. The method according to claim 5, wherein the target object is exerted with a same number of mechanical vibrations in each group of sub-measurement

8. The method according to claim 5, wherein a group of different vibration signals is generated based on a same drive signal in each group of sub-measurement.

9. The method according to claim 1, further comprising performing on the target object with a plurality of measurements, wherein in each of the plurality of measurements, mechanical vibrations are exerted on the target object based on a plurality of different vibration signals, each vibration signal corresponds to one ultrasonic echo signal; and
said acquiring elasticity parameter(s) and viscosity parameter(s) of the region of interest comprises: calculating the elasticity parameter(s) and the viscosity parameter(s) based on multiple groups of measured elasticity values and measured viscosity values, each group of measured elasticity value and measured viscosity value being calculated based on a plurality of ultrasonic echo signals acquired in each measurement.

10. The method according to claim 9, wherein during performing the plurality of measurements on the target object, a number and/or waveforms of the vibration signals used in each measurement are different.

11. The method according to claim 5, wherein the elasticity parameter is equal to a weighted average of part or all of multiple measured elasticity values, or equal to one of the multiple measured elasticity values; and the viscosity parameter is equal to a weighted average of part or all of multiple measured viscosity values, or equal to one of the multiple measured viscosity values.

12. The method according to claim 5, wherein said displaying elasticity parameter(s) and viscosity parameter(s) of the region of interest comprises:
displaying the multiple groups of measured elasticity values and measured viscosity values.

13. The method according to claim 4, wherein during each measurement performed on the target object, at least one of the following parameters of each drive signal for the plurality of different vibration signals is different: frequency, amplitude, phase and a number of periods, and at least one of the following parameters of the different vibration signals is different: frequency, amplitude, phase and a number of periods.

14. The method according to claim 4, further comprising receiving an instruction for performing each measurement inputted by a user that at least includes viscoelasticity measurement.

15. The method according to claim 1, wherein after being exerted with mechanical vibrations based on one vibration signal to acquire a corresponding ultrasonic echo signal, the target object is exerted with mechanical vibrations based on another vibration signal after a predetermined cooling time.

16. The method according to claim 1, further comprising:
while displaying the elasticity parameter(s) and the viscosity parameter(s) of the region of interest, displaying ultrasonic image(s) that is generated based on the first ultrasonic echo signal and/or the second ultrasonic echo signal.

17.-38. (canceled)

39. An ultrasonic viscoelasticity measuring apparatus, comprising:
an ultrasonic probe comprising a vibrator and a transducer, the vibrator being configured for driving the transducer to vibrate to generate a shear wave propagating in a depth direction inside a target object; the transducer comprising a plurality of array elements, at least part of the array elements being configured for transmitting a first ultrasonic wave to the target object, receiving an echo of the first ultrasonic wave and acquiring a first ultrasonic echo signal based on the echo of the first ultrasonic wave before the transducer is vibrated, and at least transmitting a second ultrasonic wave to a region of interest of the target object, receiving an echo of the second ultrasonic wave and acquiring a second ultrasonic echo signal based on the echo of the second ultrasonic wave after the transducer is vibrated;
a transmitting/receiving sequence controller configured for outputting a first transmitting/receiving sequence to the transducer before the transducer is vibrated to control the transducer to transmit the first ultrasonic wave, receive the echo of the first ultrasonic wave and acquire the first ultrasonic echo signal based on the echo of the first ultrasonic wave, outputting different drive signals to the vibrator after the region of interest is determined to control the vibrator to drive the transducer to exert various mechanical vibrations on the target object based on at least two different vibration signals, and at least outputting a second transmitting/receiving sequence to the transducer after the transducer is vibrated to control the transducer to transmit the second ultrasonic wave, receive the echo of the second ultrasonic wave and acquire the second ultrasonic echo signal based on the echo of the second ultrasonic wave;
a processor configured for generating an ultrasonic image based on the first ultrasonic echo signal, acquiring a region of interest on the ultrasonic image, and acquiring elasticity parameter(s) and viscosity parameter(s) of said region of interest based on the second ultrasonic echo signal of the region of interest under various mechanical vibrations; and
a display unit configured for displaying the elasticity parameter(s) and the viscosity parameter(s) of said region of interest.

40.-41. (canceled)

42. The apparatus according to claim 39, wherein the processor is configured for controlling to perform on the target object with one measurement, wherein in said one measurement, mechanical vibrations are exerted on the target object based on a plurality of different vibration signals, each vibration signal corresponds to one ultrasonic echo signal; and
said acquiring elasticity parameter(s) and viscosity parameter(s) of said region of interest comprises: calculating a group of elasticity parameter and viscosity parameter based on a plurality of ultrasonic echo signals corresponding to the plurality of different vibration signals.

43. The apparatus according to claim 39, wherein the processor is configured for controlling to perform on the target object with one measurement comprising multiple groups of sub-measurements, wherein in each group of sub-measurement, mechanical vibrations are exerted on the target object based on a plurality of different vibration signals, each vibration signal corresponds to one ultrasonic echo signal; and
said acquiring elasticity parameter(s) and viscosity parameter(s) of said region of interest comprises:
calculating the elasticity parameter(s) and the viscosity parameter(s) based on multiple groups of measured elasticity values and measured viscosity values, each group of measured elasticity value and measured viscosity value being calculated based on a plurality of ultrasonic echo signals corresponding to the plurality of different vibration signals in each group of sub-measurement;
or, calculating to acquire multiple groups of elasticity parameters and viscosity parameters based on a plurality of ultrasonic echo signals corresponding to the plurality of different vibration signals in each group of sub-measurement.

44.-47. (canceled)

48. The apparatus according to claim 39, wherein during performing the plurality of measurements on the target object, a number and/or waveforms of the vibration signals used in each measurement are different.

49.-61. (canceled)
